(19) United States
(12) Patent Application Publication
(10) Pub. No.: US 2014/0352412 A1
(43) Pub. Date: Dec. 4, 2014

(54) METHOD AND APPARATUS FOR MEASUREMENT AND CONTROL OF PROCESS PARAMETERS

(71) Applicant: Metafix Inc., Lachine (CA)

(72) Inventors: John RIVIERE, Lachine (CA); Denis BRUNELLE, Pointe-Claire (CA)

(21) Appl. No.: 14/287,584

(22) Filed: May 27, 2014

Related U.S. Application Data
(60) Provisional application No. 61/827,787, filed on May 28, 2013.

(51) Int. Cl. G01N 1/34; G01N 33/00

(52) U.S. Cl. CPC G01N 1/34; G01N 33/00
USPC 73/64.56

(57) ABSTRACT

A method and apparatus for measurement and control of parameters are described. The method comprises the steps of: taking a sample from each of a plurality of samplings points in a controlled sequence, the sample having a sample volume; transferring the sample volume to an instrument measurement cell according to the controlled sequence the instrument measurement cell comprising at least one instrument, and a casing having a cell volume, wherein the at least one instrument measures a value of at least one process parameter of the sample volume, and logging and/or transmitting the value of the at least one process parameter of the sample volume. The apparatus comprises: a plurality of fluid transporters, an instrument measurement cell comprising a casing having a cell volume; and at least one instrument within the casing; a manifold fluidly connected between the plurality of fluid transporters and the instrument measurement cell, and an apparatus controller.

### FIELD OF THE INVENTION

The present invention relates to the measurement and control of multiple process parameters in process industries, and particularly in the process, printing and wastewater assessment industries.

### BACKGROUND ART

In process industries, when measurements of multiple parameters at multiple points of control are required, as in the monitoring and control of a printing press parameters, the usual method is to install probes/instruments for each parameter to be measured at each of the points of measurement. This therefore requires the installation of both signal and power wiring, for each probe/instrument which is cumbersome and expensive. The multiple probes/instruments also require regular calibration and maintenance in proportion to their number.

The present method and apparatus simplifies multipoint measurements by significantly reducing the number of instruments. This method and apparatus described herein also simplifies the cleaning and calibration of the probe/instrument.

### SUMMARY

It is therefore an aim of the present invention to provide a simplified method and apparatus for the measurement and control of multiple parameters at multiple points of measurement.

Therefore, in accordance with one aspect of the present invention, there is provided a method for measurement and control of parameters, the method comprising the steps of: taking a sample from each of a plurality of samplings points in a controlled sequence, the sample having a sample volume; transferring the sample volume to an instrument measurement cell according to the controlled sequence the instrument measurement cell comprising at least one instrument, and a casing having a cell volume, wherein the at least one instrument measures a value of at least one process parameter of the sample volume, and logging and/or transmitting the value of the at least one process parameter of the sample volume.

In another aspect of the method herein described, wherein the sample volume is equal to the cell volume plus a volume within tubing connecting each of the plurality of sampling points and the instrument measurement cell.

In yet another aspect of the method herein described, wherein the controlled sequence further includes a calibration sequence comprising taking a calibration volume of reference standard solutions, and calibrating each of the least one instrument.

In still another aspect of the method herein described, wherein the process parameter is at least one of conductivity, pH, and temperature.

In yet still another aspect of the method herein described, wherein the cell volume is 100 to 250 ml.

In a further aspect of the apparatus herein described, wherein the controlled sequence further includes a washing/purging sequence comprising taking a washing solution fluid and transferring the washing solution through the instrument measurement cell.

In accordance with another aspect of the present invention, there is provided an apparatus for measurement and control of at least one process parameter from each of a plurality of sampling points, the apparatus comprising: a plurality of fluid transporters withdrawing and transferring a sample from each of the sampling points to an instrument measurement cell, the sample having a sample volume, the instrument measurement cell comprising a casing having a cell volume; and at least one instrument within the casing obtaining a measured value for the at least one process parameter of the sample volume; a manifold fluidly connected between the plurality of fluid transporters and the instrument measurement cell, and an apparatus controller stopping and starting each of the plurality of fluid transporters in a controlled sequence and transferring the sample volume according to the control sequence from each of the sampling points to the instrument measurement cell, and the apparatus controller further logging and/or transmitting the measured value.

In yet a further aspect of the apparatus herein described, further comprising at least two reference standard solution supplies; at least two reference standard solution fluid transporters for withdrawing and transferring a calibration volume of the at least two reference standard solution to the instrument measurement cell, and the apparatus controller stopping and starting the at least two reference standard solution fluid transporters in a calibration sequence, calibrating each of the least one instrument.

In still another aspect of the apparatus herein described, further comprising at least one wash solution fluid supply; the at least one wash solution fluid supply transferring a wash/purge volume of the wash solution fluid to the instrument measurement cell.

In yet still a further aspect of the apparatus herein described, wherein the at least one process parameter is at least one of conductivity, pH, and temperature.

In one embodiment of the apparatus herein described, wherein the at least one process parameter is all of the conductivity, the pH, and the temperature.

In another embodiment of the apparatus herein described, wherein the sample volume is equal to the cell volume plus a volume of tubing connecting each of the plurality of sampling points and the instrument measurement cell.

In yet another embodiment of the apparatus herein described, wherein the cell volume is 100 to 250 ml.

In yet still another embodiment of the apparatus herein described, wherein the plurality of fluid transporters are positive displacement pumps.

In a further embodiment of the apparatus herein described, wherein the positive displacement pumps are peristaltic pumps.

In yet a further embodiment of the apparatus herein described, wherein the at least two reference standard solution fluid transporters are positive displacement pumps.

In still a further embodiment of the apparatus herein described, wherein the at least one wash solution fluid supply is pressurized water.

In accordance with a further aspect of the present invention, there is provided an instrument measurement cell for measuring a process parameter of a sample volume from each of a plurality of sampling points comprising a casing having a cell volume; and at least one instrument within the casing, wherein each of the at least one instrument a measures a value of at least one process parameter of the sample volume.

In yet still a further embodiment of the apparatus herein described, wherein the at least one process parameter is at least one of conductivity, pH, and temperature.

In still a further embodiment of the apparatus herein described, wherein the at least one process parameter is all of the conductivity, the pH, and the temperature.

### BRIEF DESCRIPTION OF THE DRAWINGS

Reference will now be made to the accompanying drawings, showing by way of illustration a particular embodiment of the present invention and in which:

FIG. 1 is a schematic representation of a flow pattern used for measurement and control of parameters according to one embodiment of the present invention; and

FIG. 2 is a process flow diagram of a system/apparatus for measurement and control of parameters according to another embodiment of the present invention;

FIG. 3 illustrates a cross-sectional view of the instrument measurement cell according to an embodiment of the present invention; and

FIG. 4 illustrates a flow sheet of the portion of the system/apparatus for controlling process parameters according to one embodiment of the present invention.

### DETAILED DESCRIPTION OF PARTICULAR EMBODIMENTS

The present invention describes an improved method and apparatus for the measurement and control of process parameters, particularly conductivity, pH, and temperature in industry, particularly in the printing and water treatment industries.

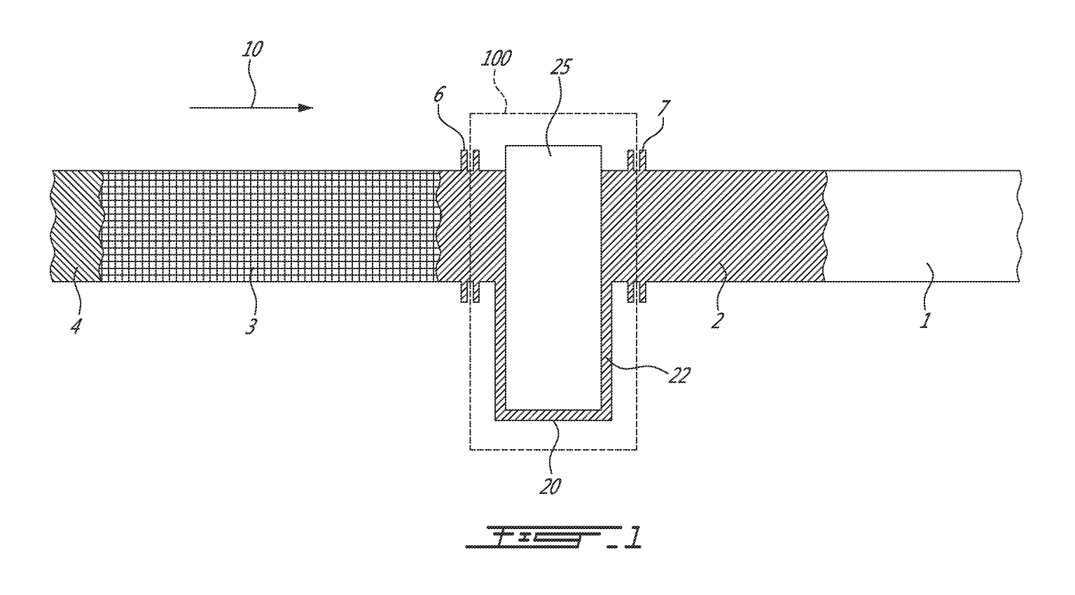

Referring now to FIG. 1 there is a schematic representation of a flow pattern used for measurement and control of process parameters according to one embodiment of a method of present invention.

### Definitions

An “instrument measurement cell” 100 is defined herein a casing having a relatively small cell volume that includes at least one instrument measuring 25 at least one process parameter. In a preferred embodiment the cell includes a plurality of instruments that may be one or more probes. In a particularly preferred embodiment the parameters measured includes all of conductivity, pH and temperature.

A “controlled sequence” is understood to be a sampling regime that withdraws a sample volume from a plurality of sampling points (multipoints) and transfers them to an instrument measurement cell be measured in a sequence established by a controller programmed by a user. The controlled sequence also includes a calibration sequence and a washing/purging sequence. A sample volume is understood to be equal to or greater than the cell volume. The sample volume the equal to the cell volume plus a volume of tubing between the sampling point and the instrument measurement cell.

A “calibration sequence” is understood as the withdrawal of a known calibration volume of one or more reference standard solutions that are used to calibrate the at least one instrument in the instrument measurement cell 100, whereby ensuring the accuracy of the value measurement of the process parameter by each of the instruments.

A “washing/purging sequence” is defined herein as the transfer of water or other washing solution to clean the instrument measurement cell 100 and the probes/instruments therein and whereby cleaning the at least one instrument and the casing of the instrument measurement cell.

The “plurality of sampling points” are the source of a plurality of samples that are taken/withdrawn and transferred by a positive displacement pump from each of the plurality of sampling points. In a particularly preferred embodiment the positive displacement pumps are peristaltic pumps. The positive displacement pumps perform two operations. On the suction side of the pump they withdraw the sample to be measured, while on the pressure side of the pump they transfer the sample from the plurality of point to the instrument measurement cell 100. These positive displacement pumps have the further advantage that with a given number of revolutions of the pump a given volume is transferred.

In the center of FIG. 1 is a measurement instrument 25 that is typically within an instrument measurement cell 100 (here represented schematically by a dotted line). The instrument 25 measures a process parameter and is enclosed within a casing 20. The casing 20 is illustrated in FIG. 1 to have a finite cell volume 22 defined by the space between the instrument 25 and an inner wall of the casing 20 and between connections 6 and 7 within the dotted lines of the instrument measurement cell 100. This finite cell volume 22 is in a preferred embodiment kept to a minimum. This minimum sample volume to be analyzed relates to the cell volume 22, and must be greater than the cell volume 22, so as to obtain a correct measured value of the process parameter being analyzed.

Generally, if the casing 20 has a cell volume of 100 ml, the volume of a sample passing through the casing 20 and around the instrument 25 is approximately 1 (80%); preferably at least 2, more preferably 3 and most preferably 5 times the cell volume. That is, the sample volume passing through the cell with a volume of 100 ml, is at least 100 ml, is preferably at least 200 ml, or 300 ml, or 500 ml.

In a preferred embodiment the cell volume 22 is a relatively small volume and is from 50 ml to 500 ml; in another embodiment the cell volume 22 is between 100 ml and 250 ml and in a preferred embodiment 100 ml to 150 ml, in a particularly preferred embodiment the cell volume 22 is 130 ml. Clearly smaller cell volumes are preferred. The cell volume may be reduced further and thereby reducing the sample volume required to passed through the casing 20 to obtain a stable measurement.

The volume of the sample that must flow through the casing 20, (i.e. a sample flow rate) is determined by the amount of fluid required to purge, a previous sample 1 (illustrated in FIG. 1) that has passed through the casing 20. To ensure a stable and accurate value of the process parameter to be measured of the sample 2 within the casing 20, the inventors have discovered that the sample volume should be at least the cell volume 22. However, in a preferred embodiment, the sample volume is the cell volume 22 plus the volume of tubing between each of the plurality of sampling points and the instrument measurement cell 100.

For a printing process parameter measurement application, ¼ inch tubing is generally used that has a volume of approximately 30 ml per 1 m length of ¼ inch tubing. Therefore, depending on the installation and the length between sampling points, the sample volume withdrawn varies. In printing installations, the length of tubing is generally 5 m and 10 m from the sampling point to the instrument measurement cell 100.

The samples to be measured are arranged to flow continuously, smoothly and in series over the instrument 25 through the casing 20. The flow direction of the series of samples is represented by the arrow 10.

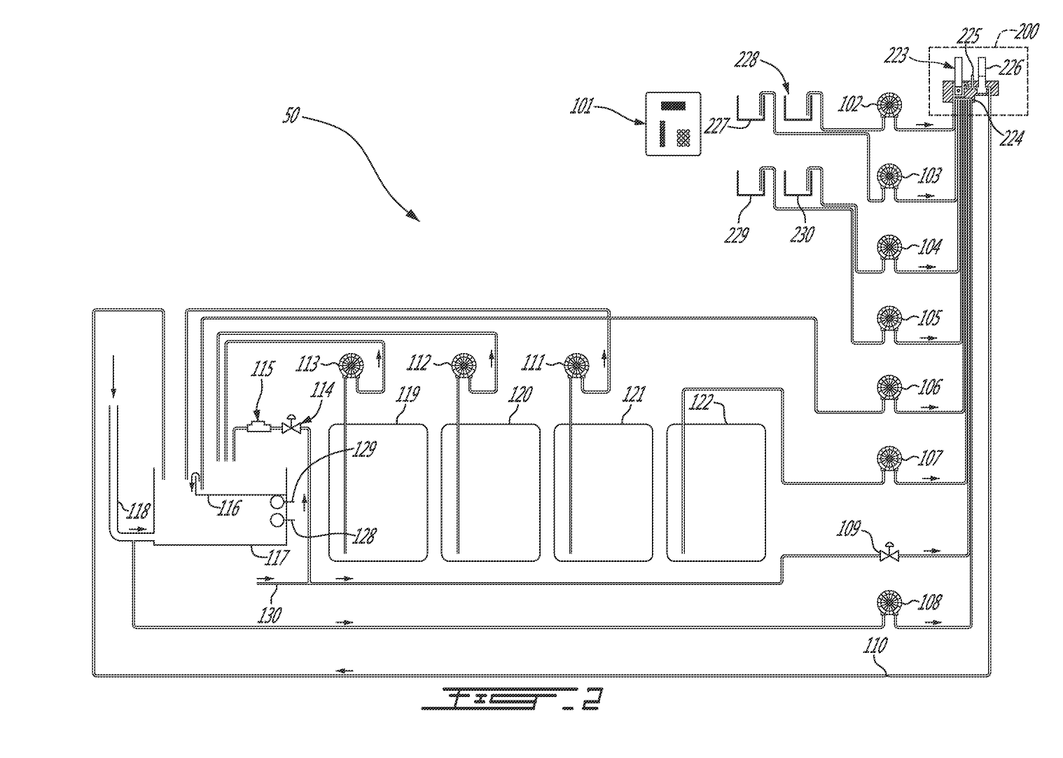

A sample 1, on the right hand side of the instrument measurement cell 100 in FIG. 1 is a previous sample that has already passed through the casing 20 and its process parameter has been measured and logged by a controller 101 (in FIG. 2). A second sample 2 is within the casing 20 and most of the sample 2 is already downstream of the instrument 25. The first portion of sample 2 is used as a purging volume for sample 1. When at least 50% of the volume of sample 2 has passed through the casing 20 the parameter for sample 2 is measured and logged by the controller 101. This sequence will continue in the flow direction illustrated by arrow 10, for samples 3 and 4 on the left of FIG. 1 and illustrated as moving towards the casing 20.

The method described herein uses as few as one instrument 25 for each parameter being measured. In a preferred embodiment more than one parameter measurement instrument 25 is regrouped into a single instrument (pH and T for example). However, in a preferred embodiment more than one process parameter is measured and therefore requires more than one instrument 25.

FIG. 2 illustrates one embodiment of a system/apparatus 50 according to the method described. At least one probe or single instrument is presented in fluid connection with the process being measured via a series of small pumps, preferably positive displacement pumps and more preferably peristaltic type that draw or suction the liquid (analyte) from each sampling point to the instrument/cell.

The method and apparatus/system 50 described herein avoid many problems common to multiple probes scattered around a press, such as encumbrance causing a lack of space to install probes, long runs of wiring, and discrepancy between probe calibration. Advantages of the method and apparatus described herein include cost effective process parameter measurement, a central location for the electronics components and probes that can be at a distance from the machinery, i.e. printing presses and thus in a more suitable location from the point of view of operation and maintenance, and uses fewer instruments.

In one embodiment of the apparatus/system 50 of the present method for conducting the measurement of various process parameters includes: an apparatus controller 101, a plurality of liquid transporters such as sampling pumps herein described as peristaltic pumps 102, 103, 104, 105, 106, 107 and 108, that draw samples having a predetermined sample volume flowrate from a plurality of sampling points 227, 228, 229 and 230, wherein a preferred embodiment are press augers. Pumps 102, 103, 104, 106, 107 and 108 have a volumetric flowrate between 250 and 750 ml/min, and preferably 300 to 500 ml/min, and most preferably 400 ml/min.

Other optional liquid transporters may also complement the operation of the system, these include solenoid valves 109 and 114 for make-up water 130 supplied from a deionised system or possibly from another pressurized water source such as a municipality. With such liquid transporters, flow restrictors 110 and flow meter 115 may also be used. Solenoids and flow regulators are generally only used for making up fresh solutions or for cleaning and flushing the system.

The apparatus 50 also includes at least one instrument. The instrument measurement cell 200 illustrated in FIG. 2 with a conductivity probe 223, grounding electrode 225, a pH probe 226 and a cell body 224. These probes 223 and 226 also include a temperature probe (not illustrated). In a preferred embodiment two conductivity probes 223 may be installed for greater confidence and to add redundancy to the instrument 200 and the apparatus 50.

In a preferred embodiment the apparatus 50 also may include more liquid transporters in the form of metering peristaltic pumps 111, 112 and 113 that go to produce more solution from an etching solution buffer tank 116, a concentrate solution storage tank 119, additive solution storage tank 120, and a recycled solution storage tank 121 and if their quality is assured they can be returned (not illustrated) to the press augers 227, 228, 229 and 230. A conductivity pH calibration solution tank 122 may also be included so that measurements obtained by the instrument probes 223 and 226 for conductivity and pH respectively can be compared with the known value of the standard conductivity solution stored in tank 122, and thus used to calibrate the cell 223 and 226 of the instrument 200.

The function of each of the components will be described in greater detail. The controller 101 may be a computer that receives electronic signals of measurements from probes 223, 225, 226, records or logs all parameter measurement data, the controller 101 may transmit the values of the collected data via an FTP (File Transfer Protocol) server. The controller 101 controls various process outputs such as sequence of stopping or starting the plurality of pumps and valves in order to maintain parameters determined by the press operator.

The fountain solution (F-S) and etching solution are herein defined as equivalents and as an aqueous liquid used in offset printing to moisten a non-image area of the plate, so that the ink is not deposited on the moistened non-image plate area.

The apparatus 50 described in FIG. 2 includes a plurality of sampling pumps 102, 103, 104 and 105 each withdrawing fountain solution by suctioning the solution from a sampling point, press augers 227, 228, 229 and 230. The number and the type of pumps will vary as a function of the number of sampling points requiring measurement and control. However, in this method each sampling point is served by a single positive displacement pump, that is designed to withdraw a predetermined sample volume that can be adjusted through the controller 101. In a preferred embodiment the sampling pumps are peristaltic pumps, although other positive displacement pumps such as gear and lobe pumps may also be used.

The pumps 102, 103, 104 and 105 transfer a predetermined sample volume of an analyte solution comprising fountain solution to an instrument/cell 200 for measurement of process parameters such as: conductivity, temperature, pH, density, viscosity, oxidation/reduction potential (ORP), surface tension, refractive index and chemical composition. In a preferred embodiment the process parameters measured by the instrument/cell 200 are: conductivity, temperature and pH.

The apparatus 50 may further include a peristaltic pump 106 withdrawing a sample volume from newly prepared F-S/etching solution. The pump 106 suctions the newly prepared F-S/etching solution from the solution buffer tank 116, and transfers the solution to the cell 200 for measurement of conductivity, temperature, pH. The system 50 may also include at least one peristaltic pump 107 that withdraws a sample volume of conductivity and pH calibration solution from tank 122, and transfers these standardized solution to the cell 200 for measurement of conductivity and pH. The measurement is used for verification of the precision and accuracy of probe and/or for calibration.

The apparatus may also include sampling peristaltic pumps 108 that suction a recirculated etching solution from pipe 118, (etching solution from press return) and transfer the solution to the cell 200 for measurement of conductivity, temperature and pH. This recirculated solution is mixed with water dosed from the solenoid valve 109 that controls incoming water 130 (from the city or from a plant treatment system i.e. deionised water system.) and may also be used for cleaning of probes 223, 226, electrode 225, and the internal passages of cell body 224. This water may also be used for the purpose of calibration of conductivity probe 223.

In a preferred embodiment, the tube downstream of the cell 200 may include a vena contracta or a flow restrictor 110, that is placed in the downstream tubing to create a backpressure when liquid is pumped. This back pressure increases the draw of electrical current by the pump when a greater back pressure is present, this increase in pump current can be monitored by the controller 101. This is an indirect method of confirming that the pump is actually drawing liquid, not air which may when liquids are withdrawn by in this manner.

Recycled pump 111 pumps recycled etching fluid optionally stored in recycled tank 121 to recirculated solution tank 117. Various additives such as alcohol (concentrated ethanol), alcohol substitutes, or pH adjusters may also be mixed in freshly prepared F-S solution by additive pump A 112 that pumps additive A from an additive storage tank 120 to etching solution buffer tank 116. In a further preferred embodiment concentrate solution pump 113 pump F-S concentrate from a F-S concentrate storage tank 119 to etching solution buffer tank 116. Solenoid valve 114 as previously described controls incoming water 130 (city or plant treatment system) to etching solution buffer tank 116 being mixed with any of the flows from tanks 119, 120 and 121. The flow from the solenoid is measured by flow meter 115 that monitors the quantity of water transferred to etching solution buffer tank 116. The etching solution tank 116 may function to co-mingling or mix the various solution throughout the apparatus and specifically the etching solutions ingredients (water, concentrate, additives). Secondly tank 116 serve as a sampling point for either operator or automated measurements of the process parameters via pump 106.

The instrument may preferably include a grounding electrode 225 that is used to force an electrical potential to ground level to avoid ground loop problems with the pH probe 226.

Clearly the conductivity probe 223 and pH probe 226 measure conductivity and pH of liquid inside cell 224 respectively.

The liquid cell 224 may be designed as a manifold accepting all the various tubes from the plurality of pumps of the apparatus 50. In a preferred embodiment the tubes being accepted at a manifold at the liquid cell 224 are small diameter tubing having a diameter between 2 and 10 mm, where 6 mm diameter tubing is preferred.

The recirculated etching solution tank 117 generally includes an operating level switch 128 at which the liquid of recirculated etching solution is normally maintained in the system. The tank 117 also may include a high level switch 129 if the volume rises rapidly and that will trigger an alarm condition.

In a preferred embodiment the cell 224 is designed for minimum cell volume of liquid around the probes, that produce faster response times and also to require less liquid to be displaced. The preferred cell volumes were previously discussed. As previously described in a preferred embodiment the liquid cell 224 has multiple inlets to avoid the use of multiple external fittings that increase system liquid volume, and consequently increase the response time which is clearly undesirable. In a preferred embodiment the liquid cell 224 includes an internal liquid routing that is designed to purge trapped air bubbles, without operator intervention.

The controller 101 integrates the operation of the apparatus. The controller ensures that the apparatus measures and controls the process parameters of interests, records or logs the values obtained and transmits data, and alert the press operator of abnormal situations. The controller 101 operates the sequence of the sampling pumps turning them on and off in a predetermined order to measure the parameters in the press augers 227, 228, 229, and 230, the press return, the etching solution tank 116, water feed 130, and any calibration solutions (if needed). With the process parameters measured, the system scans the level switches and will prepare a batch if the operating level switch 128 is low. It should be noted that the duty of transporting the F-S solution to the augers is by recirculation pumps that are not illustrated and are part of the press' support equipment. The batch F-S is made from a specific mixture of water, concentrate, additives, recycled solution. The quantity is determined by parameters and the settings of the controller 101. After a batch, the system logs the measured data and the batch data, and sends the data to a FTP server.

If an abnormal situation is found, the controller also logs this information with the data is sent to a FTP server where further consideration of the abnormal situation may be conducted.

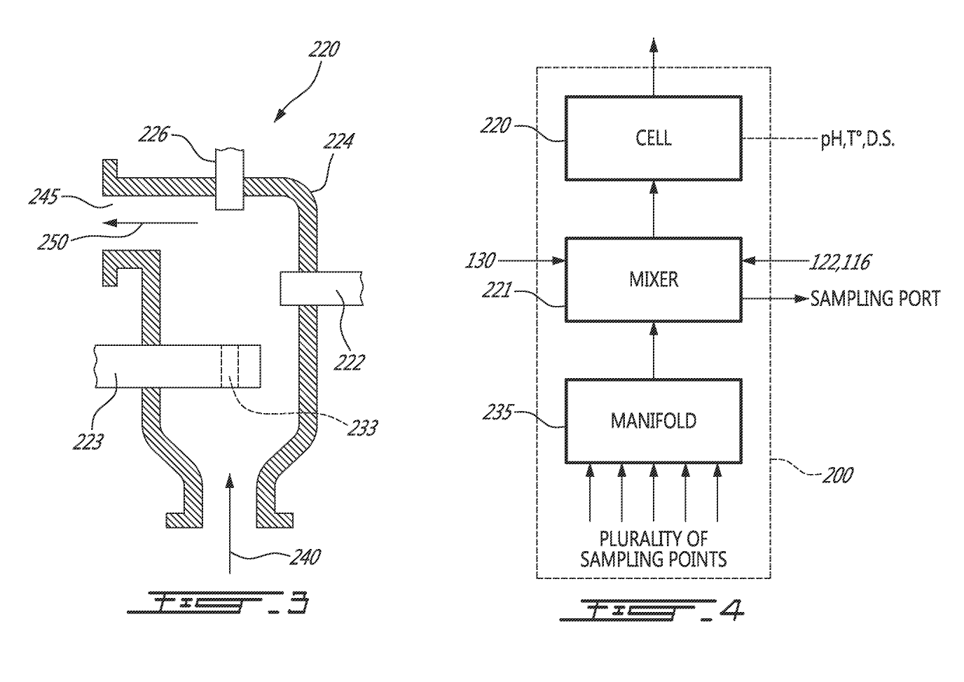

FIG. 3 illustrates a schematic cross-sectional view of the instrument measurement cell 220 of the present invention, comprising a conductivity probe 223 having an inlet channel 233 oriented in the direction of the inlet flow (illustrated by arrow 240); a temperature probe 222 and a pH probe 226. The probes 222, 226 are located near or adjacent the casing 224 inner wall.

The instrument measurement cell 220 in a preferred embodiment is made of a low friction polymer selected from the group consisting of Teflon® and polyethylene. In a preferred embodiment, the polyethylene is high density polyethylene (HDPE). The Applicant has discovered that when one of these polymers is used, a grounding electrode as illustrated in FIG. 2 was found not to be required. Although not wishing to be restricted to a theory, it is thought that by insulating the instruments from electrical inference (i.e. electrical motors, etc.), their accuracy is improved and the grounding electrode is no longer needed.

The flow outlet 245 is such that the flow direction 250 out of the cell 220 is perpendicular to the inlet direction 240. In a preferred embodiment, the cell 220 includes rounded corners facilitating the flow of the fluid through the cell.

FIG. 4 illustrates a schematic flow sheet of the system/apparatus 200 of the present invention measuring a plurality of continuously flowing samples. The plurality of samples enters the bottom of a manifold 235. Each sample is sequentially transferred to the instrument measurement cell 220 and optionally through a static mixer 221. The static mixer 221 can be the point of entry into the cell for the fountain (etching solution 116, calibration reference solution 122 and water purge 130). The mixer is also a point at which, in a preferred embodiment there is a physical sampling point for manually monitoring samples flowing through the mixer towards the cell.

The cell as previously disclosed in a preferred embodiment measures pH dissolved solids and temperature.

The embodiments of the invention described above are intended to be exemplary. Those skilled in the art will therefore appreciate that the foregoing description is illustrative only, and that various alternate configurations and modifications can be devised without departing from the spirit of the present invention. Accordingly, the present invention is intended to embrace all such alternate configurations, modifications and variances which fall within the scope of the appended claims.

1. A method for measurement and control of parameters, the method comprising the steps of:
taking a sample from each of a plurality of samplings points in a controlled sequence, the sample having a sample volume;
transferring the sample volume to an instrument measurement cell according to the controlled sequence
the instrument measurement cell comprising
at least one instrument, and
a casing having a cell volume,
wherein the at least one instrument measures a value of at least one process parameter of the sample volume, and
logging and/or transmitting the value of the at least one process parameter of the sample volume.

2. The method of claim 1, wherein the sample volume is equal to the cell volume plus a volume within tubing connecting each of the plurality of sampling points and the instrument measurement cell.

3. The method of claim 1, wherein the controlled sequence further includes a calibration sequence comprising
taking a calibration volume of reference standard solutions, and
calibrating each of the least one instrument.

4. The method of claim 1, wherein the process parameter is at least one of conductivity, pH, and temperature.

5. The method of claim 1, wherein the cell volume is 100 to 250 ml.

6. The method of claim 1, wherein the controlled sequence further includes a washing/purging sequence comprising
taking a washing solution fluid and
transferring the washing solution through the instrument measurement cell.

7. An apparatus for measurement and control of at least one process parameter from each of a plurality of sampling points, the apparatus comprising:
a plurality of fluid transporters withdrawing and transferring a sample from each of the sampling points to an instrument measurement cell, the sample having a sample volume,
the instrument measurement cell comprising
a casing having a cell volume; and
at least one instrument within the casing obtaining a measured value for the at least one process parameter of the sample volume;
a manifold fluidly connected between the plurality of fluid transporters and the instrument measurement cell, and
an apparatus controller stopping and starting each of the plurality of fluid transporters in a controlled sequence and transferring the sample volume according to the control sequence from each of the sampling points to the instrument measurement cell, and the apparatus controller further logging and/or transmitting the measured value.

8. The apparatus of claim 7, further comprising
at least two reference standard solution supplies;
at least two reference standard solution fluid transporters for withdrawing and transferring a calibration volume of the at least two reference standard solution to the instrument measurement cell, and
the apparatus controller stopping and starting the at least two reference standard solution fluid transporters in a calibration sequence, calibrating each of the least one instrument.

9. The apparatus of claim 7, further comprising
at least one wash solution fluid supply;
the at least one wash solution fluid supply transferring a wash/purge volume of the wash solution fluid to the instrument measurement cell.

10. The apparatus of claim 7, wherein the at least one process parameter is at least one of conductivity, pH, and temperature.

11. The apparatus of claim 10, wherein the at least one process parameter is all of the conductivity, the pH, and the temperature.

12. The apparatus of claim 7, wherein the sample volume is equal to the cell volume plus a volume of tubing connecting each of the plurality of sampling points and the instrument measurement cell.

13. The apparatus of claim 12, wherein the cell volume is 100 to 250 ml.

14. The apparatus of claim 7, wherein the plurality of fluid transporters are positive displacement pumps.

15. The apparatus of claim 14, wherein the positive displacement pumps are peristaltic pumps.

16. The apparatus of claim 8, wherein the at least two reference standard solution fluid transporters are positive displacement pumps.

17. The apparatus of claim 9, wherein the at least one wash solution fluid supply is pressurized water.

18. An instrument measurement cell for measuring a process parameter of a sample volume from each of a plurality of sampling points comprising
a casing having a cell volume; and
at least one instrument within the casing,
wherein each of the at least one instrument a measures a value of at least one process parameter of the sample volume.

19. The instrument measurement cell of claim 18, wherein the at least one process parameter is at least one of conductivity, pH, and temperature.

20. The instrument measurement cell of claim 19, wherein the at least one process parameter is all of the conductivity, the pH, and the temperature.
